SEQUENCE LISTING (2 sequences)

SEQ ID NO: 1 - protein, 4 aa, Artificial Sequence
  Furin-like protease cleavage site
  MISC_FEATURE (2)..(2): X = any amino acid
RXRR

SEQ ID NO: 2 - protein, 4 aa, Artificial Sequence
  Furin-like protease cleavage site
  MISC_FEATURE (2)..(2): X = any amino acid
RXKR

(19) United States
(12) Patent Application Publication
(10) Pub. No.: US 2020/0407418 A1
(43) Pub. Date: Dec. 31, 2020

(54) Cell-Mediated Exosome Delivery

(71) Applicant: Evox Therapeutics Ltd, Oxford (GB)

(72) Inventor: Joel NORDIN, Stockholm (SE)

(21) Appl. No.: 16/969,213

(22) PCT Filed: Mar. 15, 2019
(86) PCT No.: PCT/EP2019/056634

§ 371 Date: Aug. 12, 2020

(30) Foreign Application Priority Data
Mar. 16, 2018 (GB) 1804291.1

(51) Int. Cl. C07K 14/705; C07K 14/47

(52) U.S. Cl. CPC C07K 14/70596; C07K 2319/60; C07K 14/4746

(57) ABSTRACT

The present invention relates to engineered cells that enable exosome-mediated delivery of therapeutic cargoes, in particular protein therapeutics and RNA therapeutics. The present invention also relates to inventive polynucleotides, polypeptides and pharmaceutical compositions.

### TECHNICAL FIELD

The present invention relates to engineered cells that enable endogenous exosome-mediated delivery of therapeutic cargoes, in particular protein therapeutics and RNA therapeutics.

### BACKGROUND ART

Most cells, if not all, release EVs, that influence neighbouring cells or cells at a distance. The three main classes of EVs are exosomes, microvesicles (MVs) and apoptotic bodies and the common denominator is that they are cell-derived vesicles enclosed by a lipid bilayer, ranging from 30-2000 nm in diameter depending on their origin. In contrast to MVs (around 200-1000 nm in diameter), which bud directly from the plasma membrane, exosomes (50-200 nm in diameter) are derived from the endo-lysosomal pathway. EVs have been isolated from most body fluids and it is increasingly evident that they play a key role not only in the regulation of normal physiological processes, such as stem cell maintenance, tissue repair and immune surveillance, but also in the pathology underlying a range of diseases. EVs exert their biological effects in a pleiotropic manner; directly activating cell surface receptors on recipient cells via protein and bioactive lipid ligands or delivering effectors including proteins and RNAs (e.g. microRNAs (miRNAs) and mRNA). Such wide ranging biological functions suggest that EVs may have innate therapeutic potential, for example in the fields of regenerative medicine and malignant diseases. In addition to the innate therapeutic capacity of EVs, increasing attention has been drawn to their ability to naturally convey RNAs and proteins into cells, potentially making them ideal non-viral drug delivery vehicles. Indeed, numerous studies have today implicated the potential of EVs for delivery of miRNAs and other exogenous macromolecular drugs. For example, the RNA-transporting capacity of exosomes and exploited it for delivery of therapeutic siRNAs (e.g. WO2010/119256). Heusermann et al., JCB, 2016, highlighted that EVs are taken up by recipient cells in a manner similar to viruses, with fast kinetics and as single vesicles and not aggregates. Hence the uptake of EVs is a rapid process that endows EVs with unique properties for delivery of macromolecular cargo to recipient cells, both in vitro and in vivo.

CAR T cells are T cells endowed with a chimeric antigen receptor, recognising antigens similar to a B-cell receptor, but with the response of a T-cell. The potential of CAR T cell treatments have been shown with the CD19 targeting CAR T cells, which eradicate large tumours and even brain metastasis have been reported to be amenable to treatment. However, for solid tumours these treatments have not been as successful. Two main hurdles prevent CAR T treatments to be efficacious in solid tumours. The first one is that despite significant investments and rigorous research an antigen that is exclusively expressed on tumour cells has been hard to identify. Since CAR T therapy is often very effective, the side effects are commonly life-threatening if the antigen is present on non-tumour cells. CD19 exists on all B-cells, however since there is immunoglobulin replacement therapy available, the patients survive without B-cells after the treatment, but this adverse events mitigation strategy is not applicable when the CAR Ts are designed to target solid tumours. The second problem is immunomodulatory mechanisms that exist in the microenvironment of solid tumours, which prevent the CAR T cells to fully activate. To overcome the disadvantage that CAR T cells only generate a T-cell response after encountering an antigen, SynNotch receptors have been developed, which take engineered cells and their response beyond existing CAR T cells (for instance as described in patent applications WO2017193059 and US20170233474). Notch receptors are evolutionary old receptors that control transcription when the receptor binds to its ligand on a neighbouring cell. The receptor is only activated if the ligand is present on another cell. If the ligand is present on the same cell, the receptor is inhibited and it is not activated by soluble ligands either. When the Notch receptor binds a ligand the confirmation of the receptor changes and this exposes several cleavage sites for proteases that cleave the protein backbone. This subsequently releases a transcription factor (TF) on the cytoplasmic side, which transcend to the nucleus and start transcription of target genes as a response to the receptor ligand interaction. For the SynNotch system the extracellular recognition domain has been exchanged to for example a scFv, nanobody or a peptide so the receptor in theory can recognise any cell surface target. Furthermore, the TF part of the receptor was engineered to include artificially derived TFs instead of the normal domain. The cell was further equipped with a sensing element responding to the artificial TF released upon antigen recognition. So far the SynNotch system has been used to deliver therapeutics upon activation in the extracellular environment, such as cytokines and antibodies. However, one issue that remains unsolved is how to make the cell secrete therapeutically active molecules that can penetrate the nearby cells and affect proteins and/or RNA in the cytoplasm or nucleus of the recipient cells and thereby increase the druggable targets immensely for the SynNotch technology and similar platforms.

### SUMMARY OF THE INVENTION

It is hence the object of this invention to generate a cell which is responsive to its environment through the production of therapeutics EVs, preferably exosomes. Furthermore, the invention aims to satisfy existing needs within the art, for instance, ensuring a modular detection system responsive to components within the extracellular environment, inducing the expression of EVs loaded with therapeutic agents, providing a cellular on/off therapeutic system which only responds in the presence of a stimulant, a means to deliver biological agents such as RNA and proteins, a way of delivering complex, cell-produced biomolecules into target cells, and, finally, a means to deliver therapeutics with cells endogenous to specific tissues.

The present invention achieves these and other objectives by genetically engineering a cell to express a chimeric polypeptide receptor which, upon binding to its target, induces the expression of gene products to be loaded into EVs. Hence we have inventively combined engineered EVs with the chimeric antigen receptor technology to create cells that secrete designer EVs upon stimulation by a defined antigen. This extend the reach of CAR-T (and other CAR-based approaches) and chimeric antigen receptor (for instance SynNotch) technologies to be able to deliver macromolecular drugs directly into the cytoplasm of recipient cells in a specific microenvironment and/or into a particular milieu in a target organ, organ system or tissue. As a result, this leads to several advantages over existing cell therapies: (1) The treatment will be highly specific, since the activation and subsequent secretion of therapeutic EVs (typically exosomes) will only occur in the presence of a defined antigen. Additionally, specificity will be further achieved from the therapeutic cargo that is loaded into the EV, such as a miRNA, which can be designed to be highly specific for a target existing only in diseased cells. (2) The specificity will abolish the unspecific side effects seen with previous cell-based treatments such as regular CAR T cell therapies. (3) Antigens that cannot be utilised for CAR T cell treatments, due to severe side effects, can now be exploited because of point 1 and 2. (4) The system will make undruggable targets druggable. In theory any gene and/or non-coding RNA can be targeted using the inventive combination of chimeric antigen receptor technology and engineered exosomes. (5) Different cell types, for instance T cells, macrophages, NK cells, DC cells, mesenchymal stromal cells, amnion-derived cells, HEK cells, etc., can be manipulated and used as active therapeutic cells. (6) The system will be very adaptable, since the therapeutic cargo molecule and/or antigen recognised by the receptor are easily interchanged. Hence the system can simply be modulated to be utilised for treatment of both cancerous and non-malignant diseases.

In a first aspect, the present invention relates to a cell genetically modified to produce a chimeric polypeptide receptor comprising (i) an extracellular recognition domain, (ii) at least one protease cleavage site, and (iii) an intracellular transcription factor. Binding of the extracellular recognition domain to its target induces proteolytic cleavage of the at least one protease cleavage site followed by endogenous transcription by the intracellular transcription factor of at least one polynucleotide encoding a gene product comprising at least one exosomal polypeptide. In a preferred embodiment, the gene product further comprises a protein of interest (POI). As a result of the presence of the exosomal polypeptide, which may be linked covalently (for instance as a fusion protein) or non-covalently to the POI, the POI will be transported into EVs, such as exosomes, and delivered to target cells.

In another aspect, the present invention relates to an extracellular vesicle (EV) produced by the genetically modified cells. These EVs, i.e. populations of EVs, comprises the gene product, which as abovementioned typically comprises at least one exosomal polypeptide fused to a POI and/or otherwise linked to a POI. In a preferred embodiment, the EV is an exosome.

In yet another aspect, the present invention relates to a recombinant expression vector comprising the polynucleotide which encodes the gene product. Furthermore, the invention relates to a gene product encoded for by the polynucleotide.

In yet a further aspect, the present invention relates to a recombinant expression vector encoding for the chimeric polypeptide receptor, i.e. the polypeptide which is displayed at least partially on the cell surface and which comprises at least the following domains: (i) an extracellular recognition domain, (ii) at least one protease cleavage site, and (iii) an intracellular transcription factor, to drive production of the gene product.

In a further aspect, the invention relates to a method of exerting a therapeutic effect, depending on the context either in vivo, ex vivo, and/or in vitro, the method comprising:
(i) introducing a recombinant expression vector comprising the polynucleotide which encodes the gene product and a recombinant expression vector encoding for the chimeric polypeptide receptor into a cell ex vivo or in vitro;
(ii) administering the genetically modified cell to an individual.

In yet another aspect, the present invention relates to a pharmaceutical composition comprising the genetically modified cells herein, and furthermore the present invention also relates to such genetically modified cells and/or a pharmaceutical composition comprising such cells for use in medicine, for instance in the prophylaxis and/or treatment of cancer, inflammatory disease, autoimmune disease, genetic diseases, infectious diseases, metabolic diseases, CNS diseases, lysosomal storage disorders, and neurodegenerative diseases.

### BRIEF DESCRIPTION OF THE FIGURES

FIG. 1: Luciferase assay based on EVs produced from activated K652 cells comprising a chimeric polypeptide receptor targeting CD19 and further comprising a polynucleotide encoding for the exosomal protein CD63 and the reporter POI NanoLuc. Following their activation by CD19, K562 cells produce CD63-NanoLuc which is subsequently loaded into EVs produced by said cells.

FIG. 2: Red to green fluorescent conversion of reporter cells following their uptake of EVs loaded with Cre recombinase, using a Cre-EV loading strategy based on the exosome protein CD63 fused via a self-cleavable intein to the Cre protein. Following their activation by CD19 antigen as used in Example 1, K562 cells produce Cre recombinase-loaded EVs. Subsequent uptake by the reporter CD19+ve MDA-MD-231 cell line demonstrates a conversion from green to red fluorescence.

FIG. 3: Cell death of p53 sensing tumour cells following their uptake of p53 mRNA. Following their activation by the CD19 antigen in Example 1, K562 cells produce p53 mRNA together with expressing from a polynucleotide a gene product comprising a RNA-binding domain of the RNA binding protein PUF for EV loading of the p53 mRNA comprising a binding site for the PUF protein. Subsequent uptake by the p53 sensing tumour cell line results in cell death as a result of translation of the p53 mRNA delivered by the exosomes.

FIG. 4: Cell death of ovarian cancer cells following their exposure to granzyme B loaded exosomes produced by supT1 cells. Interaction between a scFv of the chimeric polypeptide receptor expressed by the supT1 cells and CA-125 tumor antigen expressed on the ovarian cancer cells. Cell death occurred in a dose-dependent fashion. Granzyme B as the protein of interest was encoded by a polynucleotide encoding the POI and the exosome protein Lamp2B, which enables transport of Granzyme B into EVs produced by the supT1 cells. Black bar of FIG. 4 represents supT1 cells engineered to target CA125, and upon target engagement produce EVs comprising granzyme B, mixed with CA-125 positive cells; white bar shows supT1 anti-CA-125 granzyme B EV-producing cells mixed with CA-125 negative cells and grey bar shows supT1 anti-CA-125 cells without the polynucleotide encoding for the gene product mixed with CA-125 positive cells. Y-axis shows percent cell death for ovarian cancer cells.

FIG. 5: Immortalized CEM (acute lymphoblastic lymphoma) cells genetically engineered to target MUC1 of breast cancer cell line T47D, using a SynNotch chimeric polypeptide receptor comprising (i) an scFv against MUC1 , (ii) the SynNotch receptor core protein, and (iii) the artificial transcription factor Gal4VP64, linked to the SynNotch core protein via a protease cleavage site (S1). Upon interaction between the scFv and MUC1 on the target T47D cells, Gal4VP64 is released and activates the expression of the EV protein CD63, which is fused to a self-cleaving intein which is in turn fused to FCU1, thereby directing FCU1 into EVs where the intein is cleaved and release free FCU1. Subsequently the FCU1-loaded EVs are taken up by T47D cells and upon administration of 5-fluorouracil the cells undergo apoptosis. The black bar in FIG. 5 represents MUC1 positive T47D cells mixed with the complete scFv-SynNotch-Gal4VP64 displaying CEM cells comprising the CD63-intein-FCU1 polynucleotide construct; the white bar represents MUC1 negative T47D cells mixed with the complete SynNotch CEM cells; and, the grey bar shows MUC1 positive T47D cells mixed with CEM cells comprising only the SynNotch-Gal4VP64 portions of the chimeric polypeptide receptor but with a functional CD63-intein-FCU1 encoding polynucleotide (in the form of plasmid DNA). As can be seen in the graph the T47D cells are only going into apoptosis after 5-fluorouracil in the MUC1 positive group when mixed with fully functional SynNotch CEM cells comprising the polynucleotide encoding for the exosomal protein (CD63) and the POI (FCU1). The Y-axis shows percentage cell death of T47D cells.

FIG. 6. Primary human T cells were transduced with a chimeric polypeptide receptor comprising the following components: (i) a camelid nanobody against PSMA, (ii) the transmembrane domain of TNFR to enable display on the cell surface of the anti-PSMA camelid nanobody, (iii) the transcription factor of the Notch intracellular domain (NID) (which comprises an S2 metalloprotease cleavage site), which is released by the cleavage of an S2 site. The T cells were also engineered to comprise a NID-responsive polynucleotide encoding the gene product CD81-PUF (where PUF is an mRNA binding protein) as well as a PTEN mRNA with PUF binding sites in the 3′ UTR. The interaction between PUF and the PUF binding sites in the PTEN mRNA actively loads the mRNA into EVs when expressed after chimeric polypeptide receptor activation. FIG. 6 shows anti-PSMA+, CD63-PUF+ and PTEN mRNA+ T-cells mixed with PC3 PSMA positive cells; white bar represents anti-PSMA+, CD63-PUF+ and PTEN mRNA+T-cells mixed with PC3 PSMA negative cells, grey bar shows anti-PSMA+, CD63-PUF+ without PTEN mRNA T-cells mixed with PC3 PSMA positive cells; and, dotted bar shows CD63-PUF+ and PTEN mRNA+ only cells without the anti-PSMA chimeric receptor T-cells mixed with PC3 PSMA positive cells. As can be seen in the graph only anti-PSMA+, CD63-PUF+ and PTEN mRNA+ T cells induce an increase in cell apoptosis analysed by flow cytometer.

FIG. 7. Primary natural killer (NK) cells were transduced with an anti-CD19 single-chain antibody fused to the SynNotch receptor together with a polynucleotide encoding the CFTR protein linked with the EV protein CD81. The NK cells were mixed with HEK 293T cells and after co-culture for 3 days the NK cells were removed and the flux of I¹²⁵ was measured. The black line with triangles in FIG. 7 shows HEK cells positive for CD19 that was mixed with antiCD19-SynNotch-CFTR cells; the grey line with black boxes shows HEK cells negative for CD19 that was mixed with antiCD19-SynNotch-CFTR cells; and, the black line with empty boxes shows HEK cells positive for CD19 that was mixed with antiCD19-SynNotch cells without the CFTR-encoding polynucleotide. Y-axis is iodide¹²⁵ efflux rate (k/min) and the x-axis represents time in minutes.

### DETAILED DESCRIPTION OF THE INVENTION

The present invention relates to an engineered cell, typically a cell line or a primary cell, which is responsive to specific extracellular stimulus through a chimeric polypeptide receptor comprising an extracellular recognition domain, a protease cleavage site and a transcription factor of which activation results in the production of EVs loaded with e.g. a protein of interest, optionally in combination with another cargo molecule, for instance a therapeutic RNA cargo. Consequently, the present invention provides for a highly modifiable, targetable, and modular delivery vehicle for a very complex biological system undeliverable by other means.

For convenience and clarity, certain terms employed herein are collected and described below. Unless otherwise defined, all technical and scientific terms used herein have the same meaning as commonly understood by one of ordinary skill in the art to which this invention belongs.

The terms “extracellular vesicle” or “EV” or “exosome” shall be understood to relate to any type of vesicle that is, for instance, obtainable from a cell, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall be understood to also relate to in some embodiments extracellular vesicle mimics, cellular membrane vesicles obtained through membrane extrusion or other techniques, etc. Essentially, the present invention may relate to any type of lipid-based structure (with vesicular morphology or with any other type of suitable morphology) that can act as a delivery or transport vehicle for the ubiquitin ligase, and optionally an antibody. It will be clear to the skilled artisan that when describing medical and scientific uses and applications of the EVs, the present invention normally relates to a plurality of EVs, i.e. a population of EVs which may comprise thousands, millions, billions or even trillions or even more EVs. In the same vein, the term “population” shall be understood to encompass a plurality of entities which together constitute such a population. In other words, individual EVs when present in a plurality constitute an EV population. Thus, naturally, the present invention pertains both to individual EVs and populations of EVs, as will be clear to the skilled person. Similar reasoning naturally applies to the genetically modified cells of the present invention, i.e. that the invention relates to both individual cells and populations of such cells.

The terms “EV protein” and “EV polypeptide” and “exosomal polypeptide” and “exosomal protein” and similar are used interchangeably herein and shall be understood to relate to any polypeptide that can be utilized to transport a polypeptide construct (which typically comprises, in addition to the exosomal protein, at least one protein of interest and/or at least any other type of biomolecule of interest, typically for therapeutic applications) to a suitable vesicular structure, i.e. to a suitable EV, normally an exosome. More specifically, these terms shall be understood as comprising any polypeptide that enables transporting, trafficking or shuttling of a fusion protein construct to a vesicular structure, such as an EV. Examples of such exosomal polypeptides are for instance CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain, CD133 , CD138 (syndecan-1), CD235a, ALIX, Syntenin-1, Syntenin-2, Lamp2, Lamp2b, syndecan-2, syndecan-3, syndecan-4, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, TSG101, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, PTGFRN, SLIT2, STX3, TCRA, TCRB, TCRD, TCRG, VTI1A, VTI1B, other exosomal polypeptides, and any combinations thereof, but numerous other polypeptides capable of transporting a polypeptide construct to an EV are comprised within the scope of the present invention. Typically, in many embodiments of the present invention, at least one exosomal polypeptide is fused to at least one POI, in order to form a fusion protein which is transported to an EV which is then secreted by the genetically modified cells. Such POIs may have an inherent therapeutic effect (such as in the case of an antibody, a bispecific or multispecific antibody derivative, a bispecific T cell engager (BiTE), a cytokine, an enzyme, etc.) but they may also act as carrier proteins for other therapeutic agents, for instance an RNA molecule such as an shRNA or an mRNA. Such fusion proteins may also comprise various other components to optimize their function(s), including linkers, transmembrane domains, cytosolic domains, multimerization domains, etc. The proteins and polypeptides mentioned herein are preferable of human origin but may also be obtained from other mammals or non-mammals.

In a first aspect, the invention relates to a genetically engineered cell that comprises a chimeric polypeptide comprising an extracellular recognition domain, at least one protease cleavage site and an intracellular transcription factor (TF). Through the action of the extracellular recognition domain interacting with its target (which may typically be present on a target cell or target organ or tissue), the protease cleavage domain undergoes cleavage to release the intracellular TF. Once released, the TF activates transcription of a polynucleotide which is present in the cell and which encodes a gene product comprising at least on exosomal polypeptide, typically fused to a POI, which is subsequently transported into an EV. The TF typically binds to a specific polynucleotide regulatory element of the polynucleotide, thereby inducing the transcription of the gene product. The POI is in advantageous embodiments a therapeutic protein of interest or a protein which is capable of transporting another biomolecule produced by the engineered cell into EVs (preferably exosomes) produced by the cell in question.

In an embodiment, the gene product which is produced through endogenous transcription by the TF is an exosomal polypeptide fused to a protein of interest (POI), typically with therapeutic activity. The protein of interest may be essentially any protein, for instance: an antibody, a single-chain antibody or any other antibody derivative such as a bispecific or multispecific antibody or antibody derivative, a bispecific T cell engager (BiTE), a receptor, a cytokine such as an interleukin, an enzyme such as caspase, granzyme, Cas, Cas9, a checkpoint inhibitor, a costimulation inhibitor, a membrane transporter such as NPC-1 or cystinosine, a splicing factor, intrabodies, single chain variable fragments (scFv), affibodies, bi- och multispecific antibodies or binders, receptors, ligands, enzymes for e.g. enzyme replacement therapy or gene editing, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In one preferred embodiment, the POI is a CRISPR-associated (Cas) polypeptide, in order to enable gene editing in target cells. Alternatively, in another preferred embodiment, the Cas polypeptide may be catalytically inactive, to enable targeted genetic engineering. Yet another alternative may be any other type of CRISPR effector such as the single RNA-guided endonuclease Cpf1. Additional preferred embodiments include POIs selected from the group comprising enzymes for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alpha-N -acetyl neuraminidase, GlcNAc phosphotransferase, mucolipin1, palmitoyl-protein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other preferred embodiments, the POI may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. Further examples of POIs include a chemokine, a chemokine receptor, a chimeric antigen receptor, a cytokine, a cytokine receptor, a differentiation factor, a growth factor, a growth factor receptor, a hormone, a metabolic enzyme, a pathogen derived protein, a proliferation inducer, a receptor, a RNA guided nuclease, a site-specific nuclease, a small molecule 2nd messenger synthesis enzyme, a T cell receptor, a toxin-derived protein, a transcription activator, a transcription repressor, a transcriptional activator, a transcriptional repressor, a translation regulator, a translational activator, a translational repressor, an activating immunoreceptor, an antibody, an apoptosis in inhibitor, an apoptosis inducer, an engineered T cell receptor, an immunoactivator, an immunoinhibitor, an inhibiting immunoreceptor, an RNA guided DNA binding protein and a second binding-triggered transcriptional switch. The POIs are typically proteins or peptides of human origin unless indicated otherwise by their name, any other nomenclature, or as known to a person skilled in the art, and they can be found in various publicly available databases such as Uniprot, RCSB, etc.

In one embodiment of the invention, the gene product following endogenous transcription is an RNA-binding protein (RBP). Non-limiting examples of RNA-binding proteins include hnRNPA1, hnRNPA2B1, DDX4, ADAD1, DAZL, ELAVL4, IGF2BP3, SAMD4A, TDP43, FUS, FMR1, FXR1, FXR2, EIF4A1-3, the MS2 coat protein, Cas6, Cas9, PUF, PUF531, PUFx2, PUFeng, and other PUF domains, as well as any domains, parts or derivates, thereof. More broadly, particular subclasses of RNA-binding proteins and domains, e.g. mRNA binding proteins (mRBPs), pre-rRNA-binding proteins, tRNA-binding proteins, small nuclear or nucleolar RNA-binding proteins, non-coding RNA-binding proteins, and transcription factors (TFs) may also be included in the gene product and used to transport RNA cargo into EVs such as exosomes. Additional non-limiting examples of RNA-binding POI include small RNA-binding domains (RBDs) (which can be both single-stranded and double-stranded RBDs (ssRBDs and dsRBDs) such as DEAD, KH, GTP_EFTU, dsrm, G-patch, IBN_N, SAP, TUDOR, RnaseA, MMR-HSR1, KOW, RnaseT, MIF4G, zf-RanBP, NTF2, PAZ, RBM1CTR, PAM2, Xpo1, Piwi, CSD, and Ribosomal_L7Ae. Such RNA-binding domains may be present in a plurality, such as in the case of PUFx2, alone or in combination with others, and may also form part of a larger RNA-binding protein construct as such, as long as their key function (i.e. the ability to transport an RNA cargo of interest, e.g. an mRNA or a short RNA) is maintained. Naturally occurring RNA cargo may be loaded into such EVs and exosomes by means of the RNA-binding protein. Optionally, the cell may be further genetically modified to comprise a nucleic acid construct which encodes for and produces or over-expresses an RNA cargo.

The RNA therapeutics cargo to be transported by the RBP may be selected from for instance the following non-limiting classes of RNAs: mRNA, miRNA, shRNA, siRNA, IncRNA, ncRNA, piRNA, piwiRNA, circRNA, tRNA, rRNA, crRNA, TLR activating oligonucleotides, as well as any other RNA molecule of interest. In a further embodiment, DNA cargo may be used instead of RNA, so that a DNA molecule of interest is transported into an EV. In a further embodiment, it is advantageous for the RNA cargo of interest to comprise domains or motifs that allows for interaction with the RNA-binding POI. Said domains and motifs may be located near the 5′ or 3′ ends of the nucleotide, typically within untranslated regions (UTRs), alternatively domains and motifs further from the polynucleotide ends, potentially within coding regions. As one non-limiting example, a suitable gene product whose transcription is activated by the TF of the chimeric polypeptide may be a fusion protein between CD63, CD81, Lamp2, syndecan, Alix (i.e. a suitable exosomal protein/polypeptide) with PUF (an RNA-binding protein/polypeptide), which will bind to the appropriate intracellular RNA motif and result in the loading of the RNA molecule into the EVs produced by the cell. This cellular engineering strategy is a highly efficacious way of delivering in situ e.g. a coding RNA molecule (such as an mRNA encoding for a suitable protein of therapeutic utility) or a non-coding RNA molecule, for instance a silencing RNA molecule such as a miRNA or a shRNA, and this type of cell-mediated in situ EV delivery is a potentially transformative approach to treating malignant diseases (e.g. solid or haematological cancers) and also potentially non-malignant diseases.

In a further embodiment, the extracellular recognition domain is an antibody, an antibody derivative, a single-chain fragment, a single-chain antibody, a nanobody, a single-domain antibody, camelid antibodies such as llama antibodies, a non-antibody recognition scaffold, a peptide, a ligand for a receptor, an adhesion molecule, a receptor, a T cell receptor, a cytokine receptor, an interleukin receptor, an extracellular matrix component, or any combination thereof. ScFvs and other types of single chain-based recognition proteins, optionally derived from antibodies and antibody-like proteins, represent highly suitable extracellular recognition domains. Non-limiting examples of target antigens to which the extracellular recognition domain may bind include disease-associated antigens, e.g. cancer-associated antigens, autoimmune disease-associated antigens, pathogen-associated antigens, inflammation-associated antigens, etc. For example, the extracellular recognition domain may be specific for a cancer-associated antigen, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE, MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. The antigen can be in alternative embodiments be associated with an inflammatory or autoimmune disease, such as type 1 diabetes, multiple sclerosis, neuromyelitis optica, rheumatoid arthritis. Non-limiting examples of antigens associated with inflammatory disease include, e.g., AOC3 (VAP-1), CAM-3001, CCL11 (eotaxin-1), CD125, CD147 (basigin), CD154 (CD40L), CD2, CD20, CD23 (IgE receptor), CD25 (α chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11a), myostatin, OX-40, scleroscin, SOST, TGF beta 1, TNF-α, and VEGF-A.

In a further embodiment, genetic engineering of the cell is performed in vitro or ex vivo. The cell is genetically engineered to express the chimeric polypeptide comprising an extracellular recognition domain, at least one protease cleavage site and an intracellular transcription factor, and furthermore, engineered to comprise the polynucleotide encoding the gene product in question. The cell may be sourced from the individual to be treated, but it is also possible to utilize allogeneic cells obtainable from related, unrelated, matched or unmatched donors. The genetic engineering process can be carried out using conventional cell engineering means, including viral transduction (using e.g. lentivirus, adenovirus, or AAVs, etc.), non-viral transfection methods, electroporation, etc. Preferably, a monoclonal culture is created, isolated, propagated and screened for genetic and expression characteristics. In many instances, an immortalized cell line is a suitable starting material for further genetic engineering of the cells. Cell lines have the advantage of allowing for scalable manufacture of the engineered therapeutic cells in question, whereas starting from primary cells and tissues may offer other advantages such as maintained natural genotype, phenotype and other characteristics, and functionality of the cells.

In a further embodiment, the cell selected for genetic modification is an effector immune cell, for instance a T cell, a cytotoxic CD8+ T cell, a CD4+ T cell, a macrophage, a monocyte, or a natural killer (NK) cell. Generally, the cell may be selected from any one of the following non-limiting examples of suitable cell types: a T cell, a cytotoxic CD8+ T cell, a CD4+ T cell, a regulatory T cell, a natural killer (NK) cell, a natural killer T (NKT) cell, a B cell, a plasma cell, a dendritic cell (DC), a macrophage, a monocyte, a neutrophil, an epithelial cell, an endothelial cell, a stem cell, an MSC, a placenta-derived cell, an amnion-derived cell, umbilical cord cells, umbilical cord blood cells, a HEK cell, a neuronal cell, an astrocyte, a microglia, etc. It may be advantageous to engineered cells from particular tissue for diseases of those organs, for instance microglial cells for the treatment of Huntington's, Parkinson's or Alzheimer's disease, or hepatocytes for the treatment of Niemann-Pick Type C, HBV or HCV, etc.

In additional advantageous embodiments, particularly suitable cells of the present invention include cells with utility for the treatment of cancer and/or autoimmunity and/or immunological disorders. Such particularly advantageous cells include macrophages, monocytes, B cells, T cells, NK cells, NKT cells, and other immune system cells. For instance, engineered macrophages or monocytes, engineered NK or NKT cells and/or engineered T cells may be highly advantageous in various cancer settings, wherein such EV-producing cell sources can function as highly potent chimeric antigen receptor cells enabling in situ delivery of therapeutic EVs such as exosomes, wherein such exosomes may comprise at least one biomolecule of interest. As abovementioned, suitable biomolecules of interest for delivery by the in situ produced EVs include proteins of interest such as antibodies, single-chain antibodies or any other antibody derivatives (for instance Nanobodies, scFvs, single-domain antibodies, bispecific antibodies, trispecific antibodies, etc.), bispecific T cell engagers (BiTEs), multispecific T cell engagers, receptors, cytokines, enzymes, checkpoint inhibitors, costimulation inhibitors, RNA-binding proteins, transporters, splicing factors, transcription factors, tumor suppressors, etc., as well as various other types of biomolecular cargo molecules. As a non-limiting example, such cells may also be engineered to comprise at least one RNA cargo molecule selected from the group of non-limiting examples comprising mRNA, sgRNA, shRNA, miRNA, shRNA, siRNA, IncRNA, ncRNA, piRNA, piwiRNA, circRNA, tRNA, rRNA, crRNA and any combination thereof.

In a further embodiment, the protease cleavage domain comprises at least one protease cleavage site. The site(s) may comprise an S1, and S2 and/or an S3 cleavage site, i.e. either one, two or three or potentially even more cleavage sites. In one embodiment, the protease cleavage site comprises a heterodimerization domain comprising the S2 proteolytic cleavage site. In yet another embodiment, the S1 proteolytic cleavage site is a furin-like protease cleavage site comprising the amino acid sequence Arg-X-(Arg/Lys)-Arg, where X is any amino acid (SEQ ID Nos: 1 and 2). The S3 cleavage site is processed by the gamma-secretase complex, releasing the intracellular transcription factor from the membrane anchor. The S2 cleavage site can furthermore be modified to be cleaved by alternative proteases, such as uPA, plasmin, PSA, MMP metalloproteases, cathepsin B and thrombin. This may be achieved by mutagenesis of the receptor at the S2 cleavage site to mutate the amino acid sequence to change the protease specificity. The S2 site can furthermore be mutated to be cleaved by organ-specific or cell-specific proteases to increase the specificity of receptor activation further. The S3 cleavage site can as well be mutated to be cleaved by different proteases apart from the natural γ-secretase. The S3 site can be mutated to be cleaved by any intra-membrane protease or any intracellular protease expressed by the cell of choice.

In another embodiment, the cells herein which are genetically engineered with the chimeric polypeptide receptor and the polynucleotide encoding the POI-containing gene product may also further comprise a growth-inducing receptor. Such a growth-inducing receptor may be either constitutively expressed by the cells or may be engineered to be under the control of the TF of the chimeric polypeptide receptor so that it is triggered by antigen recognition. Such a receptor may, as an example, comprise a single-chain antibody which binds a certain cytokine or any other soluble molecule which upon binding activates an intracellular growth response. As a non-limiting example, a single-chain antibody fused to the EpoRD2 domain which is further fused with the intracellular domain of GP130, leading to cell growth upon activation by an antigen in question. The growth-inducing receptor could for example be induced by cancer-associated proteins such as PD-L1 expressed on tumour cells, IL10, TGF-beta or VEGF. This would lead to growth of the engineered cells herein (for instance T cells) and at the same time downregulate tumour associated proteins, which would further increase the therapeutic effect. In yet another non-limiting example the growth-inducing receptor can consist of a conventional chimeric antigen receptor (CAR) which upon antigen engagement induces a T-cell response which also involves cell division and growth.

In yet another embodiment, the fusion polypeptide is a chimeric Notch polypeptide comprising from N-terminus to C-terminus and in covalent linkage. Here, the extracellular recognition domain of Notch has been replaced with a recognition domain not naturally present on a Notch receptor. Furthermore, the chimeric Notch comprises a Lin-12 Notch repeat, an S2 proteolytic cleavage site, and a transmembrane domain comprising an S3 proteolytic cleavage site. Finally, the chimeric Notch comprises an intracellular transcription factor that is heterologous to the Notch regulatory region, wherein binding of the extracellular recognition domain to its target induces cleavage at the S2 and S3 protease cleavage sites, thereby releasing the intracellular transcription factor which activates transcription of the polynucleotide to produce a gene product. Following its production, the gene product is associated with EVs, to be secreted by the cell.

In yet another embodiment, the polynucleotide encoding the gene product may further comprise a transcriptional control element, responsive to the transcription factor, operably linked to the coding sequence. Key to the present invention is the functional link between the interaction between the chimeric polypeptide receptor and the activity of the EV-mediated delivery of the at least one gene product encoded for by the at least one polynucleotide which is/are under control of the transcription factor. This unique combination of CAR cell and engineered EV technology for in situ delivery in the proximity of cancer cells could transform the treatment of solid tumours, which have so far proven to be relatively intractable to e.g. CAR T and CAR NK cell therapies.

In a further embodiment, the cells may be genetically modified to produce at least two types of fusion polypeptides, wherein at least one of the (i) extracellular recognition domain, the (ii) protease cleavage site, and the (iii) intracellular transcription factor differ between the fusion polypeptides. One alternative is to utilize two (or more, for instance three or four) fusion polypeptides where the extracellular recognition domains of the fusion polypeptides are different from one another. This way the specificity of the triggering of the EV-based activity can be controlled to a greater extent, and a multitude of antigens can be used as targets of target cells. As an example, two chimeric antigen receptors present on the same engineered cells could be used to target, as non-limiting examples, (i) HER2 and IL13Ralpha2 simultaneously, (ii) CD19 and CD3, (iii) CTLA4 and PDL1, and (iv) LAG3 and PDL1, in the context of targeting various forms of haematological and solid tumours.

In a further embodiment, endogenous production of gene products (protein and optionally RNA and/or other biomolecules of interest) as well as any other required proteins/RNA may occur through the action of multiple different chimeric polypeptide receptors, releasing different intracellular transcription factors, and the binding to various specific transcriptional control sites which may control polynucleotides encoding for the same or different gene products. As a non-limiting example, following multiple chimeric e.g. Notch-based polypeptide activations, the cell produces components involved in generating exosomes that comprise CD63-PUF (an exosomal protein fused to a RNA binding protein) and an mRNA with one or more PUF-binding sequence, as well as potentially the expression of exosomal polypeptides for immuno-evasion and tissue tropism. Furthermore, it is clear that the cell may express a single copy of a single chimeric notch receptor, multiple copies of more than one chimeric receptor, multiple copies of multiple different synthetic Notch receptors, or any combination thereof.

In a further aspect, the present invention relates to an extracellular vesicle (EV) produced by the engineered cells. The produced EVs are loaded with the gene product expressed by the intracellular transcription factor of the chimeric polypeptide receptor. Furthermore, it is preferential for the cell to produce several thousand EVs, it is even more preferably for it to produce several tens of thousands of EVs, or even millions of EVs. In a further preferred embodiment, the EV produced by the cell is an exosome. As abovementioned, important to this invention is the loading of an EV-POI fusion protein and optionally RNA drug cargo into EVs, through transcription of the EV-POI gene product (i.e. expression from the polynucleotide which encodes at least an exosomal protein). Preferentially, the EV comprises multiple copies of the gene product, for instance around 10, around 100, or even around 1000 or even up to 10000 or more copies per EV.

In a further aspect, the invention relates to a recombinant expression vector comprising the polynucleotide encoding for the gene product. Said recombinant expression vector may further comprise a transcriptional control element, responsive to the transcription factor, operably linked to the coding sequence. Further, the transcriptional control element may be endogenous or heterologous to the cell, and also the coding sequence of the polynucleotide may be endogenous or heterologous to the cell. Additionally, the present invention relates to the gene product encoded for by the polynucleotide as per above. Typically, the gene product comprises an exosomal polypeptide and a POI, with the POI having either therapeutic or targeting activity itself or having e.g. transport properties to be able to interact with other biomolecules of interest (such as RNA or DNA or proteins or peptides) to transport such biomolecules into the EVs produced by the cell.

In a further aspect, the invention relates to a recombinant expression vector encoding for the chimeric polypeptide receptor of the invention, as well as the chimeric polypeptide receptors per se.

Generally, the polynucleotide constructs may be present in various types of vectors and expression constructs, for instance plasmids, mini-circles, viruses (integrating or non-integrating), linear or circular nucleic acids such as linear DNA, or single or double stranded DNA stretches, mRNA, modified mRNA, etc. These vectors and/or expression constructs may be inducible and controlled by an external factor such as tetracyclin or doxycycline or any other type of inducer. Furthermore, polynucleotide constructs comprising the polypeptides of the present invention may be present in essentially any type of EV source cell as per the above. Introduction into a cell (typically a cell culture comprising a suitable EV-producing cell type) may, as abovementioned, be achieved using a variety of conventional techniques, such as transfection, virus-mediated transformation, electroporation, etc. Transfection may be carried out using conventional transfection reagents such as liposomes, CPPs, cationic lipids or cationic polymers, calcium phosphate, dendrimers, etc. Virus-mediated transfection is also a highly suitable methodology and may be carried out using conventional viral vectors such as adenoviral, AAV or lentiviral vectors. Virus mediated transformation is particularly relevant when creating stable cell lines for cell banking, i.e. the creation of master cell banks (MCBs) and working cell banks (WCBs) of EV-producing cell sources. The creation of stable cells and cell lines may also advantageously be achieved using electroporation, lipid-based transfection, polyethyleneimine (PEI) based transfection, or any other suitable methodology for creating stably engineered cells and/or cell lines.

In a further embodiment, the cells of the present invention may be for instance primary cells or cell lines. The cells may have been immortalized using for instance hTert, SV40 T antigen, C-MYC, v-myc, E6/E7, or any other non-limiting examples of immortalization strategies.

In a further aspect, the invention relates to a method of exerting a therapeutic effect, either in vivo, ex vivo, and/or in vitro, the method comprising:
(i) introducing (a) a recombinant expression vector comprising the polynucleotide which encodes the gene product and (b) a recombinant expression vector encoding for the chimeric polypeptide receptor into a cell ex vivo or in vitro;
(ii) administering the genetically modified cell to an individual.

The therapeutic effect is surmised to be exerted by the EVs (typically exosomes) which are produced and released by the genetically engineered (modified) cells. The EVs released from the engineered cells may, as abovementioned, comprise various types of drug cargos (such as proteins or RNA cargos) which may exert the therapeutic effect on for instance the inside of target cells and/or on the outside of target cells and/or in any other suitable locale in the body, for instance in a tumour or a metastatic site or in essentially any organ or tissue.

In yet another aspect, the present invention relates to a pharmaceutical composition comprising the genetically modified cells herein, and furthermore the present invention also relates to such genetically modified cells and/or pharmaceutical compositions for use in medicine, for instance in the prophylaxis and/or treatment of cancer, inflammatory disease, autoimmune disease, genetic diseases, infectious diseases, metabolic diseases, CNS diseases, lysosomal storage disorders, and neurodegenerative diseases.

The engineered cells (typically populations thereof) may be administered to an individual systemically, locally, regionally, or directly at the required site. The cells as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the cell and EV populations as such.

In yet another aspect, the present invention pertains to pharmaceutical compositions comprising the genetically engineered cells as per the present invention. Typically, the pharmaceutical compositions as per the present invention comprise one type of therapeutic genetically engineered cell (e.g. a population of stable cells comprising a certain type of chimeric polypeptide receptor and a polynucleotide encoding the therapeutic gene product which is responsive to the transcription factor of the chimeric polypeptide receptor) formulated with at least one pharmaceutically acceptable excipient, but more than one type of genetically engineered cell population may naturally be comprised in a pharmaceutical composition, for instance in cases where a combinatorial treatment is desirable. Naturally however, as above-mentioned, a single cell or a single population of cells may comprise more than one variety of chimeric polypeptide receptor and transcriptional control element, responsive to the transcription factor, operably linked to a coding sequence. The at least one pharmaceutically acceptable excipient may be selected from the group comprising any pharmaceutically acceptable material, composition or vehicle, for instance a solid or liquid filler, a diluent, an excipient, a carrier, a cryoprotectant, an anti-aggregation substance, platelet lysate, serum albumin and in particular recombinantly produced human serum albumin, a solvent or an encapsulating material, which may be involved in e.g. suspending, maintaining the activity of or carrying or transporting the cell population from one organ, or portion of the body, to another organ, or portion of the body (e.g. from the blood to any tissue and/or organ and/or body part of interest). The dose of cells administered to a patient will depend on the number of e.g. the disease or the symptoms to be treated or alleviated, the administration route, the pharmacological action of the gene product, the inherent properties of the EVs, the presence of any targeting entities, as well as various other parameters of relevance known to a skilled person.

The cells and EVs as per the present invention may thus be used for prophylactic and/or therapeutic purposes, e.g. for use in the prophylaxis and/or treatment and/or alleviation of various diseases and disorders. A non-limiting sample of diseases wherein the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), kidney failure, heart failure or any acute or chronic organ failure and the associated underlying etiology, graft-vs-host disease, Duchenne muscular dystrophy and other muscular dystrophies, lysosomal storage diseases such as Gaucher disease, Fabry's disease, MPS I, II (Hunter syndrome), and III, Niemann-Pick disease, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumour.

It can generally be stated that a pharmaceutical composition comprising the genetically modified cells as described herein, e.g., T cells, macrophages, monocytes, NK cells, or NKT cells or any other genetically engineered cells according to the present invention, may be administered at a dosage of 10² to 10¹² cells/kg body weight, preferably 10⁵ to 10⁸ cells/kg body weight, including all integer values within those ranges. The number of cells will depend upon the ultimate use for which the composition is intended as will the type of cells included therein. For uses provided herein, the cells are generally in a volume of a litre or less, it can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, 10¹², 10¹³, or 10¹⁴ cells. In some aspects of the present invention, particularly since essentially all the administered cells will be redirected to a particular target antigen, lower numbers of cells, in the range of 10⁶/kilogram of body weight may be administered. The engineered cell compositions may be administered multiple times at various dosages levels. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIPIα, etc.).

### Experimental and Examples

Construct design and cloning: ORFs were typically generated by synthesis and cloned into mammalian expression vectors, for instance pSF-CAG-Amp. Briefly, synthesized DNA and vector plasmid were digested with enzymes NotI and SaII as per manufacturers instruction. Restricted, purified DNA fragments were ligated together using T4 ligase as per manufacturers instruction. Successful ligation events were selected for by bacterial transformation on ampicillin-supplemented plates. Plasmid for transfection was generated by ‘maxi-prep’, as per manufacturers instruction.

Cell culture and transfection: Depending on the experimental design and assays, non-viral or viral transfection was carried out to genetically engineered cells herein. Transfection and transduction was carried out in various cell culture systems, including but not limited to 2D cell culture, shaking incubators, various forms of bioreactors, hollow fiber bioreactors, Wave bags, etc. Cells were either stably or transiently modified, using for instance electroporation, cationic lipid transfection, liposomal transfection, PEI transfection, etc. For conciseness, only a few examples of suitable cells for cell engineering are mentioned herein: HEK293T cells, supT1 cells, K652 cells, T cells generally (for instance CD8+ T cells, CD4+ T cells, regulatory T cells, etc.), NK cells generally, NKT cells generally, fibroblasts, mesenchymal stromal cells (obtainable from for instance bone marrow, adipose tissue, Wharton's jelly, perinatal tissues, placenta, amnion, etc.), B cells, neutrophils, monocytes, macrophages, dendritic cells (DCs), eosinophils, neurons, astrocytes, microglia, etc. Cells were seeded as recommended by the supplier (for instance ATCC) or manufacturer.

Assays and analytics: Western blot is a highly convenient analytical method to evaluate the enrichment of POIs in EVs, for instance exosomes. Briefly, SDS-PAGE was performed according to manufacturer's instruction (Novex PAGE 4-12% gels), whereby 1×10¹⁰ EVs and 20 ug cell lysate were loaded per well. Proteins from the SDS-PAGE gel were transferred to PVDF membrane according to manufacturer's instruction (Immobilon (RTM), Invitrogen). Membranes were typically blocked in Odyssey blocking buffer (Licor) and probed with antibodies against the POI and/or the exosomal protein according to supplier's instruction (Primary antibodies—Abcam, Secondary antibodies—Licor). Molecular probes visualized at 680 and 800 nm wavelengths. For detection of various RNA species loaded into the EVs (for instance shRNA or mRNA), qPCR was typically utilized according to the manufacturers instruction.

Example 1. Expression of CD63-NanoLuc in K562 cells through CD19-dependent chimeric notch polypeptide activation.

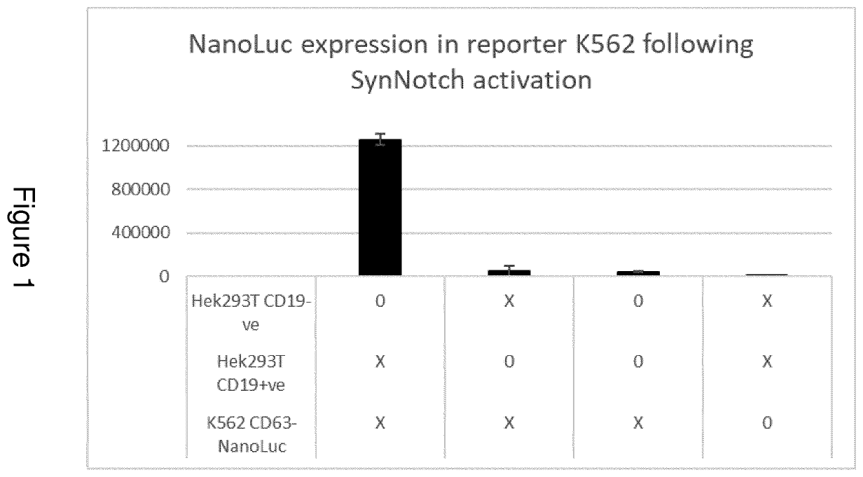

K562 cells were engineered to express a CD19-sensing chimeric Notch polypeptide, which through activation releases the intracellular transcription factor inducing the expression of CD63-NanoLuc. Furthermore, two HEK293T cell lines were generated, one with stable expression of CD19 and one without. Each variety of HEK293T cells were seeded in a 24 well plate together with the reporter chimeric Notch polypeptide K562 cells. Forty eight hours later EVs were harvested from conditioned media and measured for NanoLuc activity. Negligible amounts of NanoLuc activity were measured in samples without CD19, whereas in the presence of CD19, reporter K562 cells expressed CD63-Nanoluc in EVs. This data suggests that only through the presence of CD19 and it's interaction with chimeric Notch polypeptide on K562 cells is CD63-Nanoluc expressed and loaded into EVs. A similar experiment was carried out but this time fusing the NanoLuc reporter protein of interest to the transmembrane exosomal proteins CD81 and CD9, which belong to the tetraspanin family of proteins. These proof of principle experiment suggests that NanoLuc can be substituted with a therapeutic protein or RNA of interest to generate therapeutic effect in a target cell, tissue, organ or location (FIG. 1). Y-axis=luminescence (RLU)

Example 2. GFP expression in Cre recombinase responsive MDA cells following uptake of CD63-intein-Cre from stable K562-Chimeric Notch polypeptide cells.

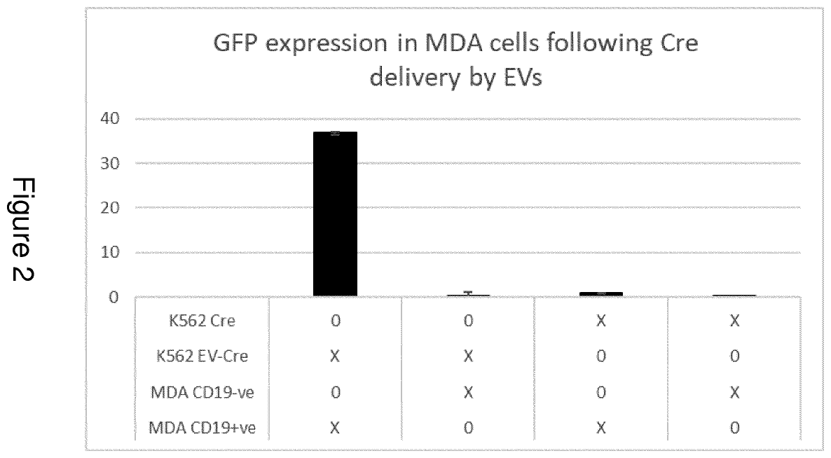

K562 cells were engineered to express a CD19-sensing Chimeric Notch polypeptide, which through its activation induced the expression of CD63-intein-Cre or soluble Cre recombinase. CD63-intein-Cre is a previously described system which allows for the loading of Cre recombinase into EVs, ultimately released from any exosomal proteins. Furthermore, an MDA-MB-231 Cre-responsive cell line was engineered to stably express CD19. Through the activity of Cre recombinase, the reporter cell has GFP expression permanently activated. Combinations of MDA-Lox+ve or -Lox-ve were co-seeded with K562-CD63-intein-Cre or -Cre in 24 well plates. Forty eight hours later, GFP+ve cells were counted against the remaining MDA population (MDA cells constitutively expresses a red fluorescent protein, which switches to green through Cre activity). In the presence of CD19 and subsequent release of EV CD63-intein-Cre, a significant shift of red to green fluorescence is observed. However, In the absence of CD19 or EV-loaded Cre, changes in fluorescence are negligible (FIG. 2). In a similar experiment, the soluble exosomal proteins Alix and syntenin were fused to the Cre recombinase via an intein, resulting in similar levels of recombination in the target MDA-MD-231 cells. Y-axis=Percent GFP positive cells.

Example 3. Cell death induced from p53 expression through EV-delivered p53 mRNA in p53 sensing cells.

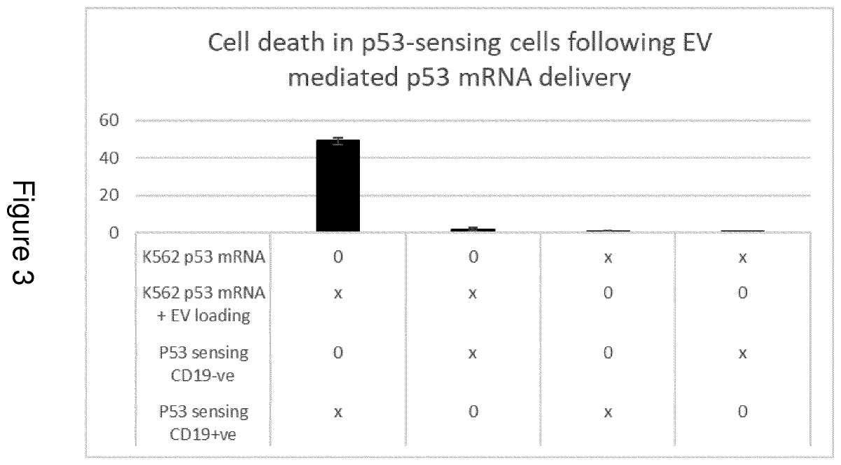

K562 cells were engineered to express a CD19-sensing SynNotch polypeptide, which through its activation induced the expression of an EV-loading-RNA-binding(CD63-PUF) polypeptide and p53 mRNA with a binding motif for the PUF protein. In addition, another K562 cell line was generated, in which p53 mRNA with a binding motif was expressed, however these cells lacked an EV loading mechanism (i.e. the fusion protein between the PUF and CD63). A tumor cell line was stably engineered to express CD19. As previous described, 48 hours later, only combinations of SynNotch activation (CD19+ve) and EV loading strategies (CD-63PUF), resulted in the delivery of p53 mRNA to the p53-sensing tumour cell line, resulting in approximately 50% death of the tumour cell line. Again, combinations lacking CD19 or CD63PUF did not result in significant cell death (FIG. 3). Y-axis=percent apoptotic cells according to annexin staining and FACS analysis.

Example 4. Cell death induced from p53 expression through EV-delivered p53 mRNA in p53 sensing cells.

K562 cells were engineered to express a mesothelin-sensing chimeric polypeptide receptor, which through its activation induced the expression of an exosomal protein-RNA-binding (CD63-PUF) polypeptide and p53 mRNA with a binding motif. In addition, another K562 cell line was generated, in which p53 mRNA with a binding motif was expressed, however lacked an EV loading mechanism. A mesothelioma cell line was stably engineered to express mesothelin. As previous described, 48 hours later, only combinations of chimeric polypeptide receptor activation (mesothelin+ve) and exosomal polypeptide (CD-63PUF), resulted in the delivery of p53 mRNA to the p53-sensing tumour cell line, resulting in approximately 50% death of the tumour cell line. Again, combinations lacking mesothelin or CD63PUF did not result in significant cell death (data not shown).

Example 5. Exosome-mediated intracellular delivery of anti-STAT3 single-chain Ab induced by chimeric polypeptide receptor binding to the TNF receptor.

K562 cells were engineered to express a chimeric polypeptide receptor comprising as its extracellular recognition domain an antibody targeting the TNF receptor. Through binding of TNFR on target immune cells in vitro, the TF of the chimeric polypeptide receptor triggered transcriptional activation and the expression of an exosomal polypeptide (syntenin) fused to a single-chain antibody fragment against intracellular target STAT3. Using a luciferase-based STAT3 sensitive cell line assay, the production and intracellular delivery of exosomes containing the syntenin-antiSTAT3-single-chain Ab was verified (data not shown).

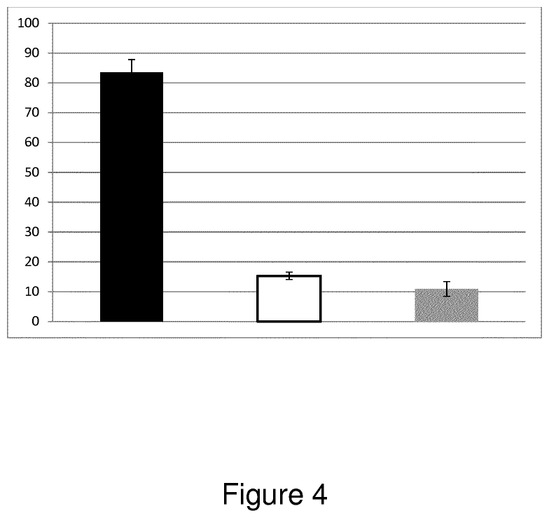

Example 6. In example 6, cell death of ovarian cancer cells (Skov3) following their exposure to granzyme B loaded exosomes produced by supT1 cells was tested. The production of exosomes loaded with granzyme B was triggered by the interaction between a scFv of the chimeric polypeptide receptor expressed by the supT1 cells and CA-125 tumor antigen expressed on the ovarian cancer cells, with Skov3 cell death occurring in a dose-dependent fashion upon exposure to increasing concentrations of the engineered supT1 cells. Granzyme B as the protein of interest was encoded by a polynucleotide encoding the POI (i.e. granzyme B) and the exosome protein Lamp2B, which enables transport of Granzyme B into EVs produced by the supT1 cells. The black bar of FIG. 4 represents supT1 cells engineered to target CA125, and upon target engagement produce EVs comprising granzyme B, mixed with CA-125 positive cells; white bar shows supT1 anti-CA-125 granzyme B EV-producing cells mixed with CA-125 negative cells and grey bar shows supT1 anti-CA-125 cells without the polynucleotide encoding for the gene product mixed with CA-125 positive cells. Y-axis shows percent cell death for Skov3 cells.

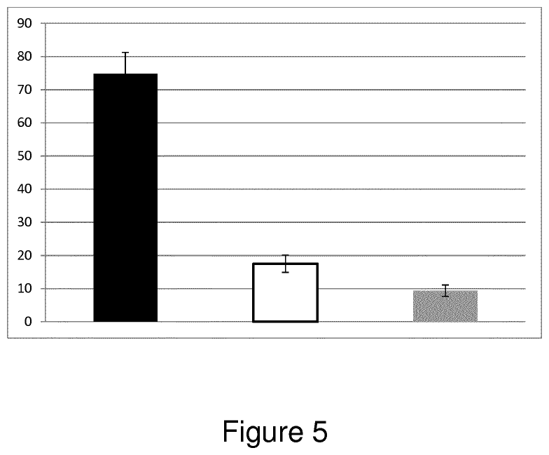

Example 7. Immortalized CEM (acute lymphoblastic lymphoma) cells were genetically engineered to target MUC1 of breast cancer cell line T47D, using a SynNotch chimeric polypeptide receptor comprising (i) an scFv against MUC1, (ii) the SynNotch receptor core protein, and (iii) the artificial transcription factor Gal4VP64, linked to the SynNotch core protein via a protease cleavage site (S1). Upon interaction between the scFv and MUC1 on the target T47D cells, Gal4VP64 is released and activates the expression of the EV protein CD63, which is fused to a self-cleaving intein which is in turn fused to FCU1, thereby directing FCU1 into EVs where the intein is cleaved and releases free FCU1. Subsequently the FCU1-loaded EVs are taken up by T47D cells and upon administration of 5-fluorouracil the cells undergo apoptosis. The black bar in FIG. 5 represents MUC1 positive T47D cells mixed with the complete scFv-SynNotch-Gal4VP64 displaying CEM cells comprising the CD63-intein-FCU1 polynucleotide construct; the white bar represents MUC1 negative T47D cells mixed with the complete SynNotch CEM cells; and, the grey bar shows MUC1 positive T47D cells mixed with CEM cells comprising only the SynNotch-Gal4VP64 portions of the chimeric polypeptide receptor but with a functional CD63-intein-FCU1 encoding polynucleotide (in the form of plasmid DNA). As can be seen in the graph the T47D cells are only going into apoptosis after 5-fluorouracil in the MUC1 positive group when mixed with fully functional SynNotch CEM cells comprising the polynucleotide encoding for the exosomal protein (CD63) and the POI (FCU1). The Y-axis shows percentage cell death of T47D cells.

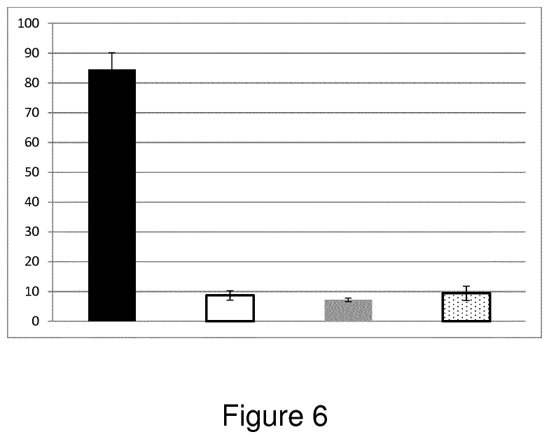

Example 8. Primary human T cells were transduced with a chimeric polypeptide receptor comprising the following components: (i) a camelid nanobody against PSMA, (ii) the transmembrane domain of TNFR to enable display on the cell surface of the anti-PSMA camelid nanobody, (iii) the transcription factor of the Notch intracellular domain (NID) (which comprises an S2 metalloprotease cleavage site), which is released by the cleavage of an S2 site. The T cells were also engineered to comprise a NID-responsive polynucleotide encoding the gene product CD81-PUF (where PUF is an mRNA binding protein) as well as a PTEN mRNA with PUF binding sites in the 3′ UTR. The interaction between PUF and the PUF binding sites in the PTEN mRNA actively loads the mRNA into EVs when expressed after chimeric polypeptide receptor activation. FIG. 6 shows anti-PSMA+, CD63-PUF+ and PTEN mRNA+ T-cells mixed with PC3 PSMA positive cells; white bar represents anti-PSMA+, CD63-PUF+ and PTEN mRNA+ T-cells mixed with PC3 PSMA negative cells, grey bar shows anti-PSMA+, CD63-PUF+ without PTEN mRNA T-cells mixed with PC3 PSMA positive cells; and, dotted bar shows CD63-PUF+ and PTEN mRNA+ only cells without the anti-PSMA chimeric receptor T-cells mixed with PC3 PSMA positive cells. As can be seen in the graph only anti-PSMA+, CD63-PUF+ and PTEN mRNA+ T cells induce an increase in cell apoptosis analysed by flow cytometer.

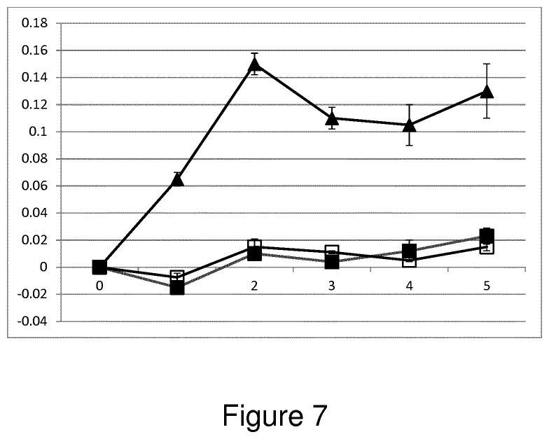

Example 9. Primary natural killer (NK) cells were transduced with an anti-CD19 single-chain antibody fused to the SynNotch receptor together with a polynucleotide encoding the CFTR protein linked with the EV protein CD81. The NK cells were mixed with HEK 293T cells and after co-culture for 3 days the NK cells were removed and the flux of I¹²⁵ was measured. The black line with triangles in FIG. 7 shows HEK cells positive for CD19 that was mixed with antiCD19-SynNotch-CFTR cells; the grey line with black boxes shows HEK cells negative for CD19 that was mixed with antiCD19-SynNotch-CFTR cells; and, the black line with empty boxes shows HEK cells positive for CD19 that was mixed with antiCD19-SynNotch cells without the CFTR-encoding polynucleotide. Y-axis is iodide¹²⁵ efflux rate (k/min) and the x-axis represents time in minutes.

1. A cell genetically modified to produce a chimeric polypeptide receptor comprising (i) an extracellular recognition domain, (ii) at least one protease cleavage site, and (iii) an intracellular transcription factor, wherein binding of the extracellular recognition domain to its target induces proteolytic cleavage of the at least one protease cleavage site and endogenous transcription by the intracellular transcription factor of at least one polynucleotide encoding a gene product comprising at least one exosomal polypeptide.

2. The cell according to claim 1, wherein the gene product further comprises a protein of interest.

3. The cell according to claim 2, wherein the protein of interest is an antibody, a single-chain antibody or any other antibody derivative, a bispecific T cell engager (BiTE), a receptor, a cytokine such as an interleukin, an enzyme such as caspase, granzyme, Cas, Cas9, a checkpoint inhibitor, a costimulation inhibitor, an RNA-binding protein, a membrane transporter such as NPC-1, a splicing factor, a protein associated with cellular organelles, a lysosomal enzyme, a transcription factor, a mitochondrial proteins, an intracellular protein, an antiviral protein, an antibacterial protein.

4. The cell according to claim 2, wherein when the protein of interest is an RNA-binding protein the cell is further genetically modified to comprise an RNA cargo molecule selected from the group consisting of mRNA, sgRNA, shRNA, miRNA, shRNA, siRNA, lncRNA, ncRNA, piRNA, piwiRNA, circRNA, tRNA, rRNA, crRNA and any combination thereof.

5. The cell according to claim 1, wherein the genetic modification is an in vitro or ex vivo genetic modification.

6. The cell according to claim 1, wherein the cell is an effector immune cell.

7. The cell according to claim 1, wherein the cell is a T cell, a cytotoxic CD8+ T cell, a CD4+ T cell, a regulatory T cell, a natural killer (NK) cell, a B cell, a plasma cell, a dendritic cell (DC), a macrophage, a monocyte, a neutrophil, an epithelial cell, an endothelial cell, a microglia, an astrocyte, a neuron, a stem cell, a bone marrow derived mesenchymal stromal cell, a Wharton's jelly derived MSC, or any other cell type.

8. The cell according to claim 1, wherein the extracellular recognition domain of the chimeric polypeptide receptor is an antibody, an antibody derivative, a single-chain fragment, a single-chain antibody, a nanobody, a peptide, a ligand for a receptor, an adhesion molecule, a receptor, an interleukin receptor, an extracellular matrix component, or any combination thereof.

9. The cell according to claim 1, wherein the at least one protease cleavage site is at least one of an S1, an S2 and/or an S3 cleavage site.

10. The cell according to claim 1, wherein the chimeric polypeptide receptor is a chimeric Notch polypeptide comprising from N-terminus to C-terminus and in covalent linkage:
(i) an extracellular recognition domain that is not naturally present in a Notch receptor polypeptide;
(ii) a Notch regulatory region which comprises a Lin 12-Notch repeat, an S2 proteolytic cleavage site, and a transmembrane domain comprising an S3 proteolytic cleavage site;
(iii)an intracellular transcription factor that is heterologous to the Notch regulatory region,
wherein binding of the extracellular recognition domain to its target induces cleavage at the S2 and S3 protease cleavage sites, thereby releasing the intracellular transcription factor which activates transcription of the polynucleotide.

11. The cell according to claim 10, wherein the Notch regulatory region further comprises a heterodimerization domain comprising the S2 proteolytic cleavage site.

12. The cell according to claim 9, wherein the S1 proteolytic cleavage site is a furin-like protease cleavage site comprising the amino acid sequence Arg-X-(Arg/Lys)-Arg, where X is any amino acid.

13. The cell according to claim 1, wherein the chimeric polypeptide receptor comprises at least one linker.

14. The cell according to claim 1, wherein the polynucleotide further comprises a transcriptional control element, responsive to the transcription factor, operably linked to a coding sequence.

15. The cell according to claim 1, wherein the cell is genetically modified to produce at least two types of chimeric polypeptide receptors, wherein at least one of the (i) extracellular recognition domain, the (ii) protease cleavage site, and the (iii) intracellular transcription factor differ between the fusion polypeptides.

16. The cell according to claim 15, wherein the extracellular recognition domains of the chimeric polypeptide receptors are different from one another.

17. An extracellular vesicle (EV) produced by the cell according to claim 1.

18. The EV according to claim 16, wherein the EV is an exosome.

19. The EV according to claim 17, wherein the EV comprises the gene product.

20. A recombinant expression vector comprising the polynucleotide, encoding for the gene product of claim 1.

21. The recombinant expression vector of claim 20, wherein the recombinant expression vector further comprises a transcriptional control element, responsive to the transcription factor, operably linked to a coding sequence.

22. The recombinant expression vector of claim 20, wherein the transcriptional control element is endogenous or heterologous to the cell.

23. The recombinant expression vector of claim 20, wherein the coding sequence of the polynucleotide is endogenous or heterologous to the cell.

24. A gene product encoded for by the polynucleotide of claim 20.

25. A recombinant expression vector encoding for the chimeric polypeptide receptor of claim 1.

26. A fusion polypeptide encoded for by the recombinant expression vector of claim 24.

27. A method of exerting a therapeutic effect, the method comprising:
(i) introducing a recombinant expression vector according to claim 20 into a cell ex vivo or in vitro; and
(ii) administering the genetically modified cell to an individual.

28. A pharmaceutical composition comprising the cell according to claim 1.

29. (canceled)

30. A cell according to claim 1 for use in the treatment of cancer, inflammatory disease, autoimmune disease, genetic diseases, infectious diseases, metabolic diseases, CNS diseases, lysosomal storage disorders, and neurodegenerative diseases.

31. A method of exerting a therapeutic effect, the method comprising:
(i) introducing a recombinant expression vector according to claim 25 into a cell ex vivo or in vitro; and
(ii) administering the genetically modified cell to an individual.
